(19) United States
(12) Patent Application Publication
(10) Pub. No.: US 2004/0242547 A1
(43) Pub. Date: Dec. 2, 2004

(54) Arylazo-substituted imidazole for the treatment of stress urinary incontinence

(76) Inventors: Robert William Gristwood, Cambridge (GB); David Cavalla, Cambridge (GB)

Correspondence Address: SALIWANCHIK LLOYD & SALIWANCHIK, A PROFESSIONAL ASSOCIATION, PO BOX 142950, GAINESVILLE, FL, 32614-2950, US

(21) Appl. No.: 10/478,977

(22) PCT Filed: Jun. 7, 2002
(86) PCT No.: PCT/GB02/02635

(30) Foreign Application Priority Data
Jun. 8, 2001 (GB) 0114008.6

(51) Int. Cl. A61K 31/655

(52) U.S. Cl. 514/150

(57) ABSTRACT

An arylazo-substituted imidazole having adrenergic alpha stimulatory activity, is useful for the treatment of stress urinary incontinence.

### FIELD OF THE INVENTION

This invention relates to a new use for known compounds.

### BACKGROUND OF THE INVENTION

Arylazo-substituted imidazoles are known. For example, U.S. Pat. No. 4,315,003 discloses 2-[(2-methoxyphenyl)azo]-1H-imidazole derivatives of the formula

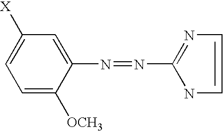

wherein X is H or halogen, and their therapeutic use in the treatment of edema, hypotension, cardiac insufficiency and mucosa hypermia. Ohnishi et al, Arzneim. Forsch/Drug Research 31 (ii): 1425-1429, discloses such a compound, i.e. 2-[(5-chloro-2-methoxyphenyl)azo]-1H-imidazole. It is described as a sympathomimetic agent resulting from direct adrenergic alpha stimulation, and Ohnishi et al conclude that it is of potential use for the treatment of hypotensive disorders.

2-[(5-Chloro-2-methoxyphenyl)azo]-1H-imidazole is currently considered to be a non-subtype-selective alpha 1 adrenoceptor agonist (hereafter “alpha agonist”; see Tocris catalogue 2001 of Tocris Cookson Ltd., Bristol, UK, Table 1, page 20). The compound is referred to in this catalogue as a hypertensive agent (page 243) and is commercially available from Tocris as a research tool. Considerations as to the selectivity of the compound are based on published studies using laboratory animal species.

Alpha 1 agonists in general cause smooth muscle contraction and thereby increase smooth muscle tone, for example in blood vessels or in the lower urinary tract. The alpha 1 receptors are currently divided into 4 subtypes, these being alpha 1A, alpha 1L (possibly a related receptor), alpha 1B and alpha 1D. Within a species, different tissues may contain a predominance of a particular alpha 1 receptor subtype. It is also known that there is a marked species dependency in the tissue distribution of adrenergic alpha 1 subtypes.

For the clinical treatment of stress urinary incontinence, efficacious agents are required which are selective for increasing the muscle tone of the lower urinary tract and which are relatively free of effects on the cardiovascular system. Recent science indicates that, for this to be achieved in the clinic, a compound with selectivity for the alpha 1A (and or 1L) receptors present in lower urinary tract (LUT) over alpha 1B receptors (present in human blood vessels) is required. Non-selective agonists will have side-effects (particularly cardiovascular), due to the activation of alpha 1B receptors, resulting in increases in vascular tone and undesirable increases in blood pressure.

Because of the species differences referred to above, studies with human tissues are currently required to see whether a compound is efficacious against human LUT smooth muscle and has selectivity in this effect compared with effects on human blood vessels. Insofar as any conclusions can or should be drawn from available data, any efficacy of 2-[(5-chloro-2-methoxyphenyl)azo]-1H-imidazole on human LUT tissues would be associated with an undesirable elevation in blood pressure.

### SUMMARY OF THE INVENTION

Surprisingly, it has now been found that 2-[(5-chloro-2-methoxyphenyl)azo]-1H-imidazole has the desired selective activity, in that it is active at increasing the tone of human LUT tissue whilst being much less active at increasing the tone of human arterial blood vessels. These data indicate that the compound will be useful in the treatment of stress urinary incontinence in man. More generally, according to the present invention, an arylazo-substituted imidazole having adrenergic alpha stimulatory activity is useful in the treatment of stress urinary incontinence.

### DESCRIPTION OF INVENTION

Any arylazo-substituted imidazole may be used in this invention, provided that it has the desired activity. Its adrenergic alpha stimulatory activity may be determined by known assays, and is preferably at least substantially as for noradrenaline. This activity is preferably at least 50% of the activity exhibited by 2-[(5-chloro-2-methoxyphenyl)azo]-1H-imidazole.

Preferred agents for use in the invention have one or more structural characteristics of the formula shown above. Most preferably, the compound is 2-[(5-chloro-2-methoxyphenyl)azo]-1H-imidazole (X=Cl).

By means of the invention, stress urinary incontinence in humans can be treated, e.g. controlled or prevented. For this purpose, the active compound can be formulated in any suitable manner, together with a conventional diluent or carrier, e.g. as a tablet or capsule, or as a sustained release formulation. The active compound may be administered by any suitable route, e.g. intravaginal (for which purpose a pessary or ring device may be used), and is preferably administered by the oral route.

Suitable compositions for oral use include tablets, troches, lozenges, aqueous or oily suspensions, dispersible powders or granules, emulsions, hard or soft capsules, syrups and elixirs. Suitable additives include sweetening agents, flavouring agents, colouring agents and preserving agents. Tablets contain the active ingredient in admixture with non-toxic pharmaceutically acceptable excipients, e.g. inert diluents such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyceryl distearate may be employed. They may also be coated, to form osmotic therapeutic tablets for controlled release. Hard gelatin capsules may include an inert solid diluent, for example calcium carbonate, calcium phosphate or kaolin; soft gelatin capsules may include water or an oil medium, for example peanut oil, liquid paraffin or olive oil.

The amount of active agent to be administered can be readily determined by the skilled person, and will depend on the usual factor such as the frequency of dosing, the age and condition of the patient, the nature and degree of the complaint, the rate of administration, coadministered drugs etc. A typical daily dosage may be 0.1-1000, e.g. 1-100, mg.

The data on which this invention is based will now be described.

Test System

Determinations were made of agonist potency on human LUT and blood vessel smooth muscle. Tissue sources: human lower urinary tract smooth muscle strips (prostatic/urethral) were obtained from patients undergoing transurethral resection of the prostate procedure for benign prostatic hyperplasia. Human mesenteric arteries (500-750 μm internal diameter) were obtained from patients undergoing bowel resection for carcinoma. Tissues were preserved in Krebs-bicarbonate solution at 4° C. from the time of surgery until used in functional experiments within 24 hours.

Protocol

Sections of mesenteric artery (5 mm in length) were set up between stainless steel stirrups after removal of the endothelium by gentle rubbing. Strips of prostatic/urethral smooth muscle (3×10 mm) and the mesenteric arteries were set up under 1 g resting tension in Krebs solution of composition (mM): NaCl 118.4; KCl 4.7; CaCl₂ 1.9; NaHCO₃ 25.0; MgSO₄ 1.2; KH2PO₄ 1.2; glucose 11.7, gassed with 5% CO₂ in O₂ and maintained at 37° C. The tension developed by all tissues was measured by means of isometric force transducers (Lectromed UFI 57G sensitivity) and recorded to computer via a Cambridge Electronic Design 1401 analogue to digital converter, using CHART software. Tissues were equilibrated for 60 minutes with several changes of bathing medium prior to starting drug administrations. All experiments were performed in the presence of cocaine (10 μM) and corticosterone (10 μM), to prevent neuronal and extraneuronal uptake of drugs, and propanolol (1 μM), to block B adrenoreceptors.

Agonist Responses

Cumulative concentration-response curves were obtained to 2-[(5-chloro-2-methoxyphenyl)azo]-1H-imidazole, and to noradrenaline for comparison.

For the LUT tissue following recording of the final drug response to noradrenaline or 2-[(5-chloro-2-methoxyphenyl)azo]-1H-imidazole, 100 μM KCl was added to indicate the maximum ability of the tissue to respond.

For the mesenteric artery preparations following the recording of the final drug response to 2-[(5-chloro-2-methoxyphenyl)azo]-1H-imidazole, a supramaximal concentration of noradrenaline was added as a further test for partial agonism.

Data Analysis

Increases in developed tension to agonist were plotted and individual EC50 values were determined. From these, pEC50 (negative log of the EC50) values were determined for each experiment and where appropriate mean values of these with standard error of the means calculated.

2-[(5-Chloro-2-methoxyphenyl)azo]-1H-imidazole was active on the LUT (appearing a full agonist compared with noradrenaline) tissue and less active on the blood vessels (appearing a partial agonist compared with noradrenaline).

On LUT, both drugs produced concentration-related increases in tension, indicating contraction of the tissue. Noradrenaline had a pEC50 of 5.4±0.39 and produced a maximum response equivalent to 63.7±4.8% of the response to 100 mM potassium chloride (n=3). 2-[(5-Chloro-2-methoxyphenyl)azo]-1H-imidazole had a pEC50 of 5.7 with a maximum response equivalent to 62.5% of the response to 100 mM potassium chloride. These data indicate that on human LUT 2-[(5-chloro-2-methoxyphenyl)azo]-1H-imidazole is at least as potent as noradrenaline with a similar maximum effect.

On mesenteric arteries, both drugs produced concentration-related increases in tension, indicating contraction of the tissue. Noradrenaline had a pEC50 of 6.18±0.32 with a mean maximum contraction of 4.22±2.21 g (n=10). 2-[(5-Chloro-2-methoxyphenyl)azo]-1H-imidazole had a pEC50 of 5.3±0.1 with a mean maximal contraction (occurring at 20 micromolar) of 1.1±0.7 g (n=3). No further increase in mean tension occurred with higher concentrations of 2-[(5-chloro-2-methoxyphenyl)azo]-1H-imidazole. These data show 2-[(5-chloro-2-methoxyphenyl)azo]-1H-imidazole to be weaker than noradrenaline on the blood vessels and to have a much lower maximum effect, consistent with 2-[(5-chloro-2-methoxyphenyl)azo]-1H-imidazole being a partial agonist on this tissue. Further evidence that 2-[(5-chloro-2-methoxyphenyl)azo]-1H-imidazole is a partial agonist compared with noradrenaline was obtained. Once the highest concentration of 2-[(5-chloro-2-methoxyphenyl)azo]-1H-imidazole had been administered and the maximum effect defined, a concentration of noradrenaline (160-210 μM) known to be capable of producing a maximum response was added to the organ bath. In all cases, large further increases in tension were produced by the noradrenaline, with a mean further increase of 2.7±0.9 g (n=3).

1. A method for treating stress urinary incontinence wherein said method comprises administering to a patient in need of such treatment an arylazo-substituted imidazole having adrenergic alpha stimulatory activity.

2. The method, according to claim 1, wherein the imidazole has the formula 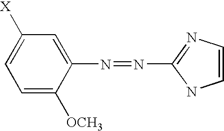
wherein X is H or halogen.

3. The method according to claim 1, wherein the imidazole is 2-[(5-chloro-2-methoxyphenyl)azo]-1H-imidazole.
